(19) United States
(12) Patent Application Publication
(10) Pub. No.: US 2019/0357792 A1
(43) Pub. Date: Nov. 28, 2019

(54) SENSIBILITY EVALUATION APPARATUS, SENSIBILITY EVALUATION METHOD AND METHOD FOR CONFIGURING MULTI-AXIS SENSIBILITY MODEL

(71) Applicant: HIROSHIMA UNIVERSITY, Higashihiroshima-shi (JP)

(72) Inventors: Masahiro MACHIZAWA, Hiroshima (JP); Shigeto YAMAWAKI, Hiroshima (JP)

(21) Appl. No.: 16/418,946

(22) Filed: May 21, 2019

(30) Foreign Application Priority Data
May 25, 2018 (JP) 2018-100273

(51) Int. Cl. A61B 5/048; A61B 5/0484; A61B 5/16; A61B 5/00

(52) U.S. Cl. CPC A61B 5/048; A61B 5/0042; A61B 5/165; A61B 5/0484

(57) ABSTRACT

A sensibility evaluation apparatus includes: a specifier configured to specify, among human types, a human type of a user; an extractor configured to specify, for each of the at least one neurophysiological index, among neurophysiological data of a user, neurophysiological data belonging to clusters of the neurophysiological data to extract at least a feature value from the neurophysiological data specified; a first evaluator configured to select, for each of the at least one neurophysiological index, a weighting coefficient corresponding to the human type of the user from predetermined weighting coefficients by the predetermined human types and apply the weighting coefficient selected to the at least one feature value extracted to evaluate the each of the at least one neurophysiological index; and a second evaluator configured to select a weighting coefficient corresponding to the human type of the user from predetermined weighting coefficients by the predetermined human types and apply the weighting coefficient selected to the each of the at least one neurophysiological index calculated to evaluate the degree.

### CROSS-REFERENCE TO RELATED APPLICATION

The present application is based on, and claims priority from JP Application Number 2018-100273 filed May 25, 2018, the disclosure of which is hereby incorporated by reference herein in its entirety.

### BACKGROUND

The present disclosure relates to an apparatus and a method for quantitative evaluation of sensibility (KANSEI) and a method for configuring a multi-axis sensibility model which is a model serving as a basis of the quantitative evaluation of the sensibility (KANSEI).

When a human operates an object such as a machine or a computer, he or she generally operates an auxiliary device, e.g., a handle, a lever, a button, a keyboard, or a mouse, with part of his or her body such as hands and feet, or communicates with the object through speech or gesture. Research and development have been made on a technology, which is called “Brain Machine Interface (BMI)” or “Brain Computer Interface (BCI)”, of directly connecting human's brain activity to a machine so that the human can operate the machine as intended. BMI or BCI is expected to improve usability of an object through direct communication between the human and the object. Also in the fields of medical care and welfare, it is expected that BMI or BCI allows people, who lost their motor function or sensory function due to an accident or disease, to operate the object at their own will so that they can communicate with other people.

If human's mental activity or information of his or her mind, such as unconsciousness or subconsciousness, in particular sensibility, could be read, human and mind-friendly objects and services would be provided. For example, if human's sensibility about an object could be objectively detected or predicted, an object that would evoke such sensibility from the human would be designed in advance. Further, the information about the sensibility thus read can improve mental care and human-to-human communication. The present inventors aim to develop a Brain Emotion Interface (BEI) which reads the human's sensibility, and achieves connection or communication between humans, or between humans and objects, using the read sensibility information.

Various approaches for quantitatively evaluating human sensibility have been proposed, many of which, however, are quantitative evaluation methods based on any fixed standard. Examples of the quantitative evaluation methods include those based on an absolute value of a heart rate or a heart rate variability and those based on neural information with reference to, for example, a variable value which is corresponding to activities in an area in the brain and which is obtained by magnetic resonance imaging (MRI) or the like and/or a variable value of power of a specific frequency obtained from a electroencephalogram (EEG) or the like. For example, US 2018/0303370 A1 discloses a method for quantitatively evaluating sensibility. The method includes: extracting neurophysiological data related to axes of a multi-axis sensibility model from regions of interest respectively relevant to pleasant/unpleasant, activation/deactivation, and a sense of expectation or anticipation, the axes including a pleasant/unpleasant axis, an activation/deactivation axis, and an anticipation axis; and evaluating the sensibility with reference to the neurophysiological data of the axes of the multi-axis sensibility model.

In the method for evaluating sensibility disclosed in US 2018/0303370 A1, it is proposed that the sensibility is quantitatively evaluated based on the multi-axis sensibility model, which is optimized participant to person. Therefore, to quantitatively evaluate sensibility of a person of unknown without a particular model has been set, an optimized model for the person has to be configured first at the time of evaluation. It indeed consumes time and labor to configure such a model person-to-person each time, and therefore, not it is not readily available evaluate sensibility of everyone from the beginning.

As a mean to solve to the problem, one could apply a single, fixed averaged model that was prepared in advance based on some individuals on a person unknown in order to quantitatively evaluate sensibility of the person. However, people are significantly different in personality due to various factors such as gender, age, and character, and it is also known that neurophysiological responses corresponding to each factor is often different from individual to individual. Therefore, when a common standard model is applied to people who are significantly different from each other in personality and neurophysiological data, results of evaluation merely achieve a low accuracy because of discrepancies between an average person and the particular individual.

### SUMMARY

An aspect of the present disclosure provides a sensibility evaluation apparatus for evaluating a degree of sensibility (KANSEI) of a person. The degree is represented by Σp×(Σq×x), where x is at least one feature value extracted from neurophysiological data measured with a neural activity measuring apparatus, q is a weighting coefficient of the at least one feature value, (Σq×x) is at least one neurophysiological index relating to the sensibility of the person, and p is a weighting coefficient of the at least one neurophysiological index. The sensibility evaluation apparatus includes: a specifier configured to specify, among predetermined human types which are obtained by classifying traits of people, a human type of a user subjected to evaluation of sensibility; an extractor configured to receive the neurophysiological data of the user measured with the neural activity measuring apparatus to specify, for each of the at least one neurophysiological index, among the neurophysiological data received, neurophysiological data which belong to predetermined clusters of the neurophysiological data received and which have statistical significance to the each of the at least one neurophysiological index, and extract the at least one feature value from the neurophysiological data specified; a first evaluator configured to select, for each of the at least one neurophysiological index, a weighting coefficient q corresponding to the human type of the user from predetermined weighting coefficients q by the predetermined human types and apply the weighting coefficient q selected to the at least one feature value extracted to evaluate the each of the at least one neurophysiological index; and a second evaluator configured to select a weighting coefficient p corresponding to the human type of the user from predetermined weighting coefficients p by the predetermined human types and apply the weighting coefficient p selected to the each of the at least one neurophysiological index calculated to evaluate the degree.

Moreover, another aspect of the present disclosure provides a sensibility evaluation method related to the sensibility evaluation apparatus.

Moreover, still another aspect of the present disclosure provides a method for configuring a multi-axis sensibility model representing a degree of sensibility (KANSEI) of a person by Σp×(Σq×x), where x is at least one feature value extracted from neurophysiological data measured with a neural activity measuring apparatus, q is a weighting coefficient of the at least one feature value, (Σq×x) is at least one neurophysiological index relating to the sensibility of the person, and p is a weighting coefficient of the at least one neurophysiological index. The method includes: clustering qualitative data representing traits of the person to determine human types for classification of the traits of the person; performing a regression analysis by the human types on subjective evaluation values of the at least one neurophysiological index obtained by performing a subjective evaluation experiment on participants to calculate weighting coefficients p by the human types; selecting, for each of the at least one neurophysiological index, among neurophysiological data of the participants measured in the subjective evaluation experiment, neurophysiological data having statistical significance to the each of the at least one neurophysiological index; clustering, for each of the at least one neurophysiological index, the neurophysiological data selected to determine clusters of the neurophysiological data; and obtaining, for each of the at least one neurophysiological index, relevance of each of the human types with respect to the clusters to convert the relevance into the weighting coefficients q by the human types.

### BRIEF DESCRIPTION OF THE DRAWINGS

FIG. 1 schematically shows relationship among emotions, feelings, and sensibility.

FIG. 2 schematically shows a multi-axis sensibility model advocated by the present inventors.

FIG. 3 shows regions of interest relevant to axes of the multi-axis sensibility model.

FIG. 4 shows various fMRI images obtained when a participant is in a pleasant state.

FIG. 5 shows an fMRI image, and a sagittal section of the brain on which EEG signal sources are plotted, both of which are obtained in the pleasant state.

FIG. 6 shows a result of a time-frequency analysis that was carried out on signals of the EEG signal sources in the region of interest (posterior cingulate gyms in the pleasant state).

FIG. 7 shows an fMRI image, and a sagittal section of the brain on which EEG signal sources are plotted, both of which were obtained when the participant is in an active state.

FIG. 8 shows a result of a time-frequency analysis that was carried out on signals of the EEG signal sources in the region of interest (posterior cingulate gyms in the active state).

FIG. 9 generally shows how to carry out an experiment of presenting participants with pleasant/unpleasant stimulus images.

FIGS. 10A and 10B show fMRI images of a subject's brain in a pleasant image expectation state and in an unpleasant image expectation state, respectively.

FIG. 11A shows a sagittal section of the brain (a region of parietal lobe) on which plotted are signal sources corresponding to a difference between EEG signals measured in a pleasant image expectation state and in an unpleasant image expectation state, and FIGS. 11B to 11D show time-frequency distributions of the EEG signals of the region.

FIG. 12A shows a sagittal section of the brain (visual cortex) on which plotted are signal sources corresponding to a difference between EEG signals measured in a pleasant image expectation state and in an unpleasant image expectation state, and FIGS. 12B to 12D show time-frequency distributions of the EEG signals of the region.

FIG. 13 shows a block flow diagram illustrating a configuration procedure of multi-axis sensibility models by human types.

FIG. 14 shows a view schematically illustrating three human types determined by the clustering of five factors of character traits.

FIG. 15 shows an example of self-evaluation for determining a subjective physiological axis.

FIG. 16 shows a flow chart for selection of independent components of an electroencephalogram in the region of interest and their frequency bands.

FIG. 17 shows components (electroencephalogram topographic images) representing signal intensity distributions of independent components extracted by an independent component analysis carried out on electroencephalogram signals.

FIG. 18 shows a sagittal section of the brain on which estimated positions of the signal sources of the independent signal components are plotted.

FIG. 19 shows a result of a time-frequency analysis carried out on signals of the EEG signal sources.

FIG. 20 shows a schematic example of scattered dots, each dot representing one of independent components observed from a person, illustrating clusters of neurophysiological data relating to pleasant/unpleasant and their electroencephalogram topographic images.

FIGS. 21A to 21C show graphs illustrating relevance of the human types with respect to the clusters for a corresponding one of neurophysiological indices of pleasant/unpleasant, activation/deactivation, and anticipation, respectively.

FIG. 22 is a block diagram illustrating a sensibility evaluation apparatus according to an embodiment of the present disclosure.

FIGS. 23A to 23C show topographical images of selected independent components of interest relevant to pleasant/unpleasant and their corresponding weighting coefficients.

FIGS. 24A to 24C are views schematically illustrating evaluated degrees of pleasant/unpleasant, activation/deactivation, and anticipation, respectively.

FIG. 25 is a view illustrating a display example of an excitement indicator.

FIG. 26 is a view schematically illustrating an embodiment in which the sensibility evaluation apparatus is placed in a cloud environment.

### DETAILED DESCRIPTION

Embodiments will be described in detail with reference to the drawings as needed. Note that excessively detailed description will sometimes be omitted herein to avoid complexity. For example, detailed description of a matter already well known in the art and redundant description of substantially the same configuration will sometimes be omitted herein. This will be done to avoid redundancies in the following description and facilitate the understanding of those skilled in the art.

Note that the present inventors provide the following detailed description and the accompanying drawings only to help those skilled in the art fully appreciate the present invention and do not intend to limit the scope of the subject matter of the appended claims by that description or those drawings.

A sensibility evaluation apparatus and a sensibility evaluation method according to the present disclosure adopt neither a model optimized for each individual (multi-axis sensibility model) nor an average single standard model applicable to everyone. In the sensibility evaluation apparatus and the sensibility evaluation method according to the present disclosure, a multi-axis sensibility model is prepared for each human type, and to quantitatively evaluate sensibility of a person unknown, a degree of the sensibility (KANSEI) of the person is evaluated and output based on a multi-axis sensibility model corresponding to a human type of the person. A method for configuring the multi-axis sensibility models by the human types, and an apparatus and a method for evaluating a degree of sensibility (KANSEI) of a person based on the multi-axis sensibility models by the human types will specifically be described below.

### 1. Definition of Sensibility

A human being feels a sense of excitement, exhilaration, or suspense, or feels a flutter, on seeing or hearing something or on touching something or being touched by something. It has been considered that these senses are not mere emotions or feelings but brought about by complex, higher neural functions in which exteroception information entering the brain through a somatic nervous system including motor nerves and sensory nerves, in a meanwhile interoception built on an autonomic nervous system including sympathetic nerves and parasympathetic nerves, memories, experiences, and other factors are deeply intertwined with each other.

The present inventors grasp these complex, higher neural functions such as the senses of exhilaration, suspense, and flutter, which are distinctly different from mere emotions or feeling, as “sensitivities (KANSEI)” comprehensively. The present inventors also define the sensitivities as a higher neural function of synthesizing together the exteroceptive information (somatic nervous system) and the interoceptive information (autonomic nervous system) and looking down upon an emotional reaction produced by reference to past experiences and memories from an even higher level. In other words, the “sensibility” can be said to be a higher neural function allowing a person to intuitively sense the gap between his or her prediction (image) and the result (sense information) by comparing it to his or her past experiences and knowledge.

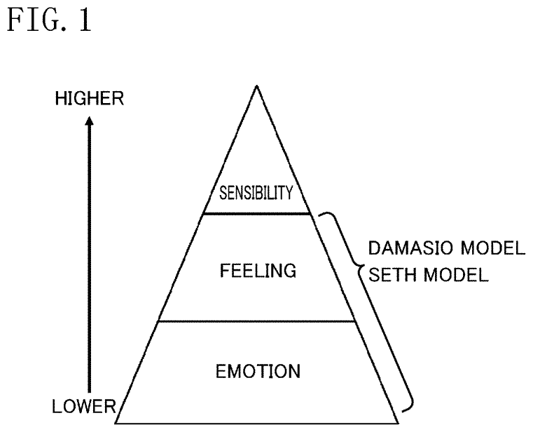

The three concepts of emotions, feelings, and sensibility will be described below. FIG. 1 schematically illustrates relationship among emotions, feelings, and sensibility. The emotions are an unconscious and instinctive neural function caused by external stimulation, and is the lowest neural function among the three. The feelings are conscientized emotions, and are a higher neural function than the emotions. The sensibility is a neural function, unique to human beings, reflective of their experiences and knowledge, and is the highest neural function among the three.

Viewing the sensibility that is such a higher neural function in perspective requires grasping the sensibility comprehensively from various points of view or aspects.

For example, the sensibility may be grasped from a “pleasant/unpleasant” point of view or aspect by determining whether the person is feeling fine, pleased, or comfortable, or otherwise, feeling sick, displeased, or uncomfortable.

The sensibility may also be grasped from an “active/inactive” point of view or aspect by determining whether the person is awakened, heated, or active, or otherwise, absent-minded, calm, or inactive.

The sensibility may also be grasped from an “anticipation” point of view or aspect by determining whether the person is excited with the expectation or anticipation of something, or otherwise, bitterly disappointed and discouraged.

A Russell's circular ring model, plotting the “pleasant/unpleasant” and “active/inactive” parameters on dual axes, is known. The feelings can be represented by this circular ring model. However, the present inventors believe that the sensibility is a higher neural function of comparing the gap between the prediction (image) and the result (sense information) to experiences and knowledge, and therefore, cannot be sufficiently expressed by the traditional circular ring model comprised of the two axes indicating pleasant/unpleasant and activation/deactivation. Thus, the present inventors advocate a multi-axis sensibility model in which the time axis (indicating anticipation, for example) is added as a third axis to the Russell's circular ring model.

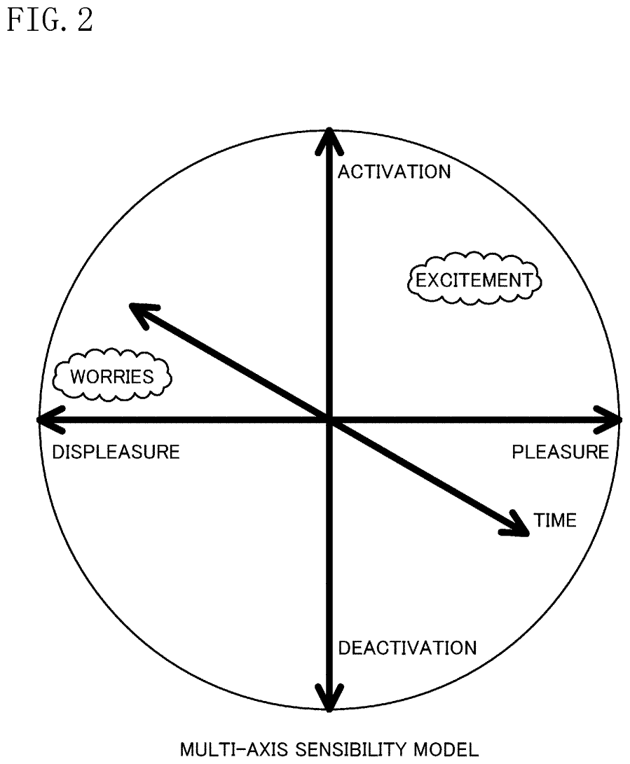

FIG. 2 schematically shows the multi-axis sensibility model advocated by the present inventors. The multi-axis sensibility model can plot, for example, a “pleasant/unpleasant” parameter on a first axis, an “active/inactive” parameter on a second axis, and a “time” (anticipation) parameter on a third axis. Representing the sensibility in the form of a multi-axis model is advantageous because values of these axes are evaluated and synthesized so that the sensibility, which is a vague and broad concept, can be quantitatively evaluated, or visualized.

Correct evaluation of the sensibility, which is the higher neural function, would lead to establishment of the BEI technology that connects humans and objects together. If the sensibility information is used in various fields, new values can be created, and new merits can be provided. For example, it can be considered that social implementation of the BEI technology is achieved through creation of products and systems that response more appropriately to human mind as they are used more frequently, and grow emotional values, such as pleasure, willingness, and affection.

### 2. Identification of Region of Interest

It will be described below the results of fMRI and EEG measurements performed to identify which part of the brain is active in the neural responses of “pleasant/unpleasant,” “activation/deactivation,” and “anticipation.” The measurement results are fundamental data for visualizing and quantifying the sensibility, and thus, are of significant importance. fMRI is one of brain function imaging methods in which a certain mental process is noninvasively associated with a specific brain structure.

fMRI measures a signal intensity depending on the level of oxygen in a regional neural blood flow involved in neural activities. For this reason, fMRI is sometimes called a “Blood Oxygen Level Dependent” (BOLD) method.

Activities of nerve cells in the brain require a lot of oxygen. Thus, oxyhemoglobin, which is hemoglobin bonded with oxygen, flows toward the nerve cells through the neural blood flow. At that time, oxygen supplied exceeds the oxygen intake of the nerve cells, and as a result, reduced hemoglobin (deoxyhemoglobin) that has transported oxygen relatively decreases locally. The reduced hemoglobin has magnetic properties, and locally produces nonuniformity in the magnetic field around the blood vessel. Using hemoglobin that varies the magnetic properties depending on the bonding with oxygen, fMRI catches signal enhancement that occurs secondarily due to local change in oxygenation balance of the neural blood flow accompanying the activities of the nerve cells. At present, it is possible to measure in seconds the local change in the neural blood flow in the whole brain at a spatial resolution of about several millimeters.

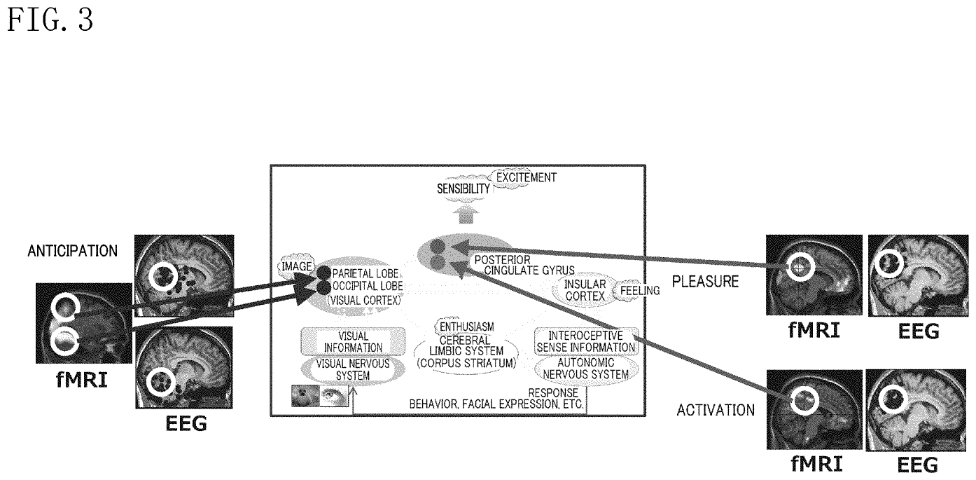

FIG. 3 shows regions of interest relevant to the axes of the multi-axis sensibility model, together with the results of fMRI and EEG measurements on the neural responses related to the axes. An fMRI image and an EEG image, which are related to the “pleasant/unpleasant” axis or the “activation/deactivation” axis in FIG. 3, respectively represent a difference (change) between signals obtained in a pleasant state and an unpleasant state, and a difference (change) between signals obtained in an active state and an inactive state. The fMRI image related to the “anticipation” axis is obtained in a pleasant image expectation state, and the EEG images respectively represent a difference between signals obtained in a pleasant image expectation state and those obtained in an unpleasant image expectation state.

As shown in FIG. 3, the results of the fMRI and EEG measurements indicate that cingulate gyrus is active when the subject feels “pleasant/unpleasant” and “active/inactive.” It is also indicated that parietal lobe and visual cortex are active when the subject feels the “anticipation.”

The regions of interest related to the axes of the multi-axis sensibility model shown in FIG. 3 have been found through observations and experiments of the neural responses under various different conditions using fMRI and EEG. The observations and experiments will be specifically described below.

(1) Neural Responses in Pleasant/Unpleasant State

First of all, a pleasant image (e.g., an image of a cute baby seal) and an unpleasant image (e.g., an image of hazardous industrial wastes), extracted from International Affective Picture System (IAPS), were presented to 27 participants to observe their neural responses when they were in a pleasant/unpleasant state.

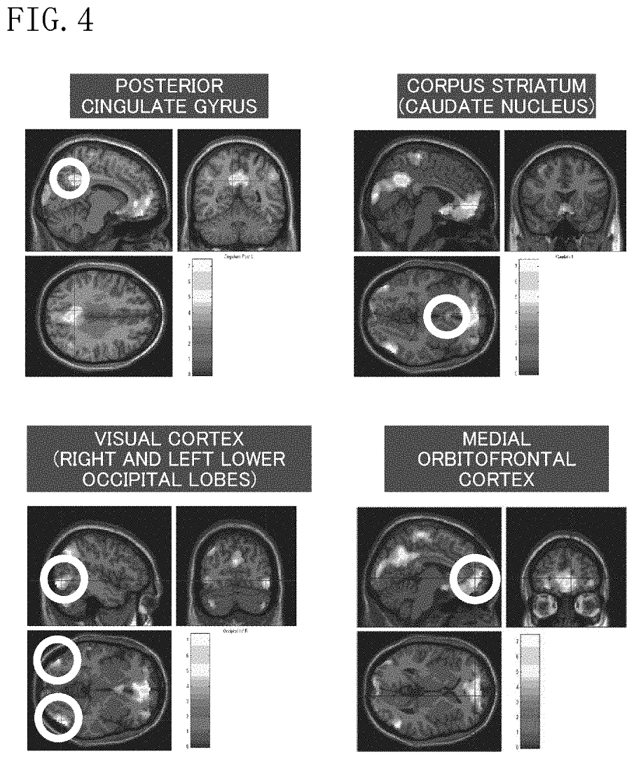

FIG. 4 shows various fMRI images (sagittal, coronal, and horizontal sections) of the brain in the pleasant state. In FIG. 4, regions that responded more significantly in a pleasant state (when the participant saw the pleasant image) than in an unpleasant state (when the participant saw the unpleasant image) are marked with circles. As can be seen from FIG. 4, posterior cingulate gyms, visual cortex, corpus striatum, and orbitofrontal area are activated in the pleasant state.

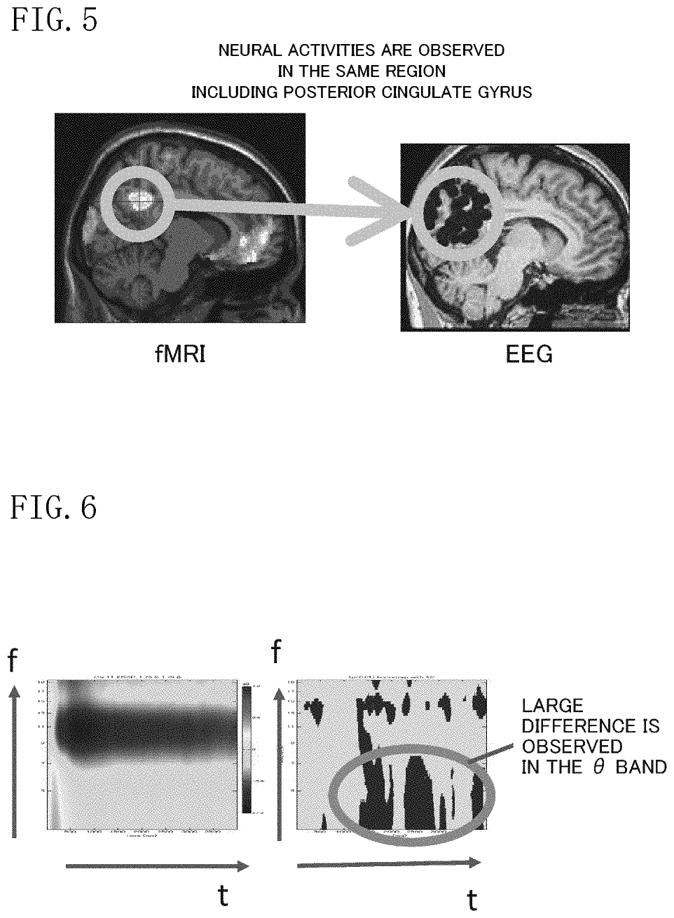

FIG. 5 shows an fMRI image, and a sagittal section of the brain on which EEG signal sources are plotted, both of which were obtained in the pleasant state. In FIG. 5, regions that responded more significantly in the pleasant state than in the unpleasant state are marked with circles. As can be seen from FIG. 5, the measurement results by fMRI and the measurement results by EEG show the neural activities in the same region including the posterior cingulate gyms in the pleasant state. According to the results, the region including the cingulate gyms can be identified as a region of interest related to the pleasant/unpleasant state.

FIG. 6 shows a result of a time-frequency analysis that was carried out on a signal of the EEG signal source in the region of interest (the posterior cingulate gyms in the pleasant state). FIG. 6 shows, on the left, a result of a time-frequency analysis that was carried out on a signal of the EEG signal source in the region of interest (the posterior cingulate gyms in the pleasant state). FIG. 6 shows, on the right, a difference between signals obtained in the pleasant state and those obtained in the unpleasant state. In the right graph of FIG. 6, dark-colored parts indicate that the difference is large. The results of the EEG measurement reveal that responses in the θ bands of the region of interest are involved in the pleasant state.

(2) Neural Responses in Active/Inactive State

First of all, an active image (e.g., an image of appetizing sushi) and an inactive image (e.g., an image of a castle stood in a quiet rural area), extracted from IAPS, were presented to 27 participants to observe their neural responses when they were in an active/inactive state.

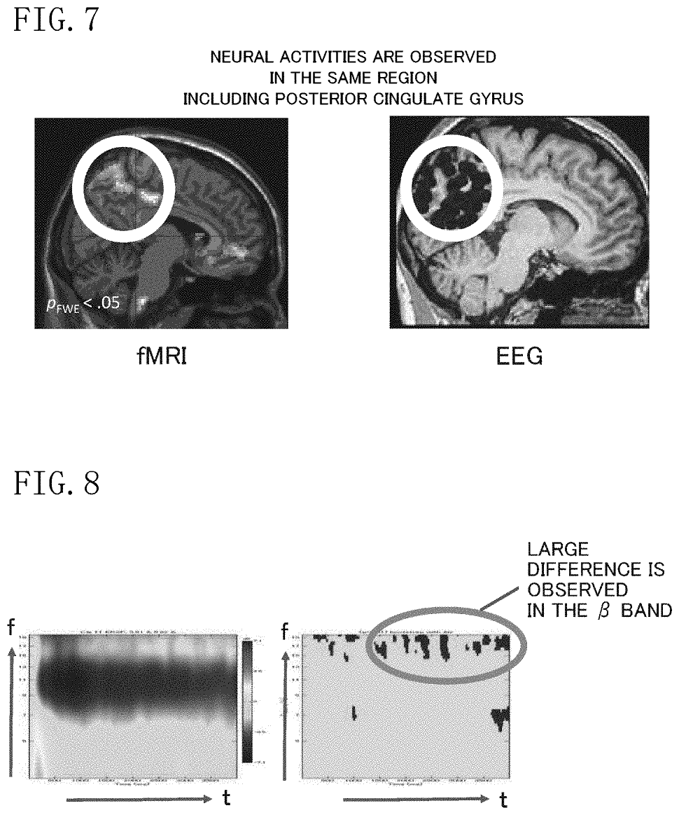

FIG. 7 shows an fMRI image, and a sagittal section of the brain on which EEG signal sources are plotted, both of which were obtained in the active state. In FIG. 7, regions that responded more significantly in the active state (when the participant saw the active image) than in the inactive state (when the participant saw the inactive image) are marked with circles. As can be seen from FIG. 7, the measurement results by fVMRI and the measurement results by EEG show the neural activities in the same region including the posterior cingulate gyms in the active state. According to the results, the region including the cingulate gyms can be identified as a region of interest related to the active/inactive state.

FIG. 8 shows a result of a time-frequency analysis that was carried out on a signal of the EEG signal source in the region of interest (the posterior cingulate gyms in the active state). FIG. 8 shows, on the left, a result of a time-frequency analysis that was carried out on a signal of the EEG signal source in the region of interest (the posterior cingulate gyms in the active state). FIG. 8 shows, on the right, a difference between signals obtained in the active state and those obtained in the inactive state. In the right graph of FIG. 8, dark-colored parts indicate that the difference is large. The results of the EEG measurement reveal that responses in the 3 bands of the region of interest were involved in the active state.

(3) Neural Responses in Anticipation

First of all, an experiment is carried out in which 27 participants are presented with stimulus images that will evoke their emotions to evaluate the feeling states of those participants who are viewing those images. As the stimulus images, 80 emotion-evoking color images, extracted from IAPS, are used. Of those 80 images, 40 are images that would evoke pleasure (“pleasant images”) and the other 40 are images that would evoke displeasure (“unpleasant images”).

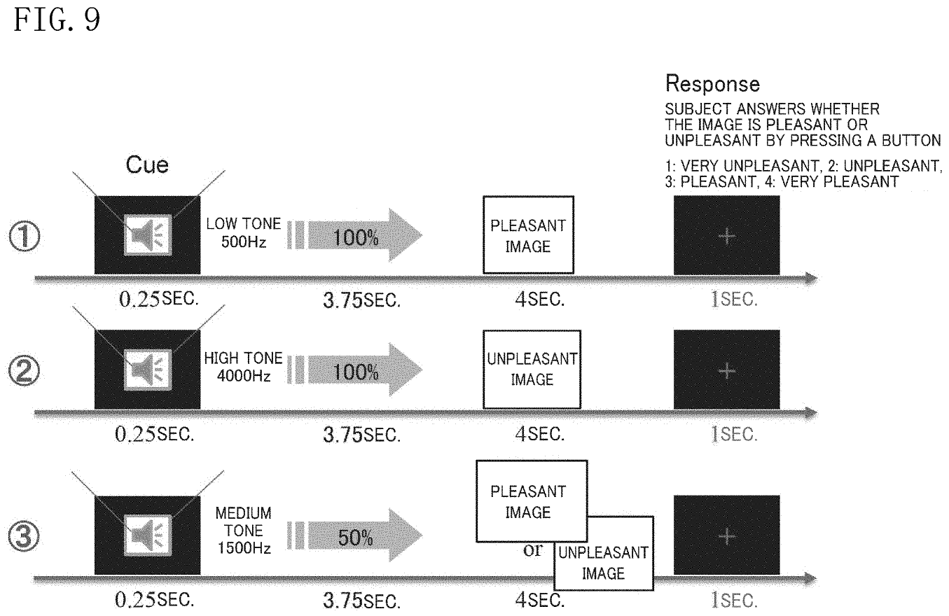

FIG. 9 illustrates generally how to carry out the experiment of presenting the participants with those pleasant/unpleasant stimulus images. Each of those stimulus images will be presented for only 4 seconds to the participants 3.75 seconds after a short tone (Cue) has been emitted for 0.25 seconds. Then, the participants are each urged to answer, by pressing the button, whether they have found the image pleasant or unpleasant. In this experiment, a pleasant image is presented to the participants every time a low tone (with a frequency of 500 Hz) has been emitted; an unpleasant image is presented to the participants every time a high tone (with a frequency of 4,000 Hz) has been emitted; and either a pleasant image or an unpleasant image is presented at a probability of 50% after a medium tone (with a frequency of 1,500 Hz) has been emitted.

In this experiment, that 4-second interval between a point in time when any of these three types of tones is emitted and a point in time when the image is presented is a period in which the participants expect what will happen next (i.e., presentation of either a pleasant image or an unpleasant image in this experiment). Their neural activities are observed during this expectation period. For example, when a low tone is emitted, the participants are in the state of “pleasant image expectation” in which they are expecting to be presented with a pleasant image. On the other hand, when a high tone is emitted, the participants are in the state of “unpleasant image expectation” in which they are expecting to be presented with an unpleasant image. Meanwhile, when a medium tone is emitted, the participants are in a “pleasant/unpleasant unexpectable state” in which they are not sure which of the two types of images will be presented, a pleasant image or an unpleasant image.

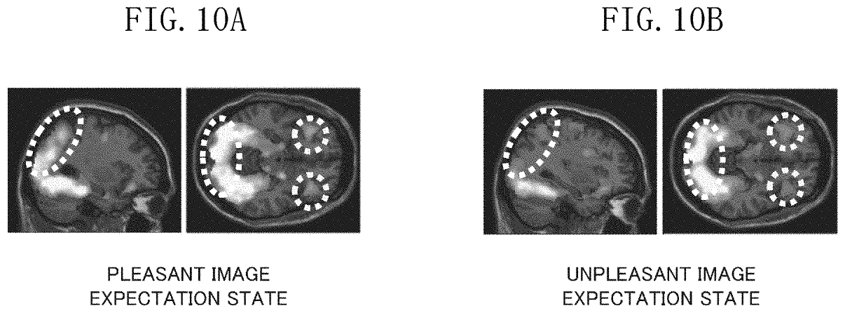

FIGS. 10A and 10B show fMRI images (representing sagittal and horizontal sections) of a subject's brain in the pleasant image expectation state and in the unpleasant image expectation state, respectively. As indicated clearly by the dotted circles in FIGS. 10A and 10B, it can be seen that according to fMRI, brain regions including the parietal lobe, visual cortex, and insular cortex are involved in the pleasant image expectation and unpleasant image expectation.

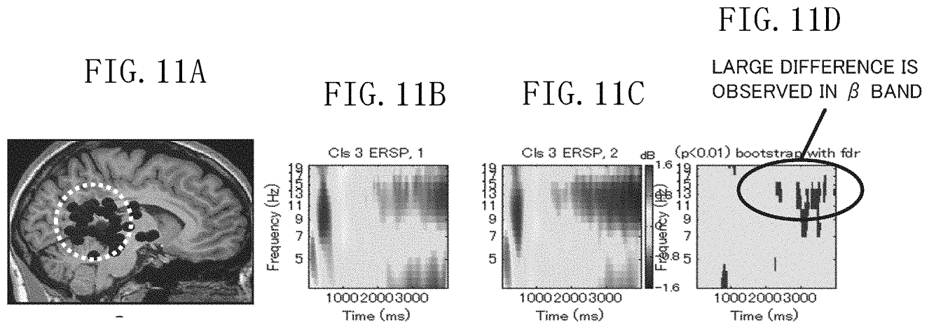

FIGS. 11A to 11D show the results of EEG measurement. FIG. 11A shows a sagittal section of a subject's brain, with a dotted circle added to a region that responded more significantly in the pleasant image expectation state than in the unpleasant image expectation state. FIG. 11B shows a result of a time-frequency analysis that was carried out on a signal of the EEG signal source in the region of interest (a region of the parietal lobe in the pleasant image expectation state). FIG. 11C shows a result of a time-frequency analysis that was carried out on a signal of the EEG signal source in the region of interest (a region of the parietal lobe in the unpleasant image expectation state). FIG. 11D shows the difference between the signals obtained in the pleasant image expectation state and the unpleasant image expectation state. In FIG. 11D, the region with a significant difference between them is encircled. Other regions had no difference. These EEG measurement results reveal that reactions in the 3 bands of the parietal lobe were involved in the pleasant image expectation.

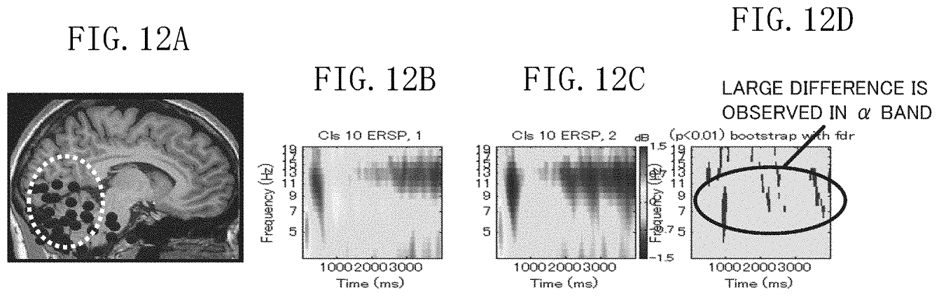

FIGS. 12A to 12D show the results of EEG measurement. FIG. 12A shows a sagittal section of a subject' brain, with a dotted circle added to a region that responded more significantly in the pleasant image expectation state than in the unpleasant image expectation state. FIG. 12B shows a result of a time-frequency analysis that was carried out on a signal of the EEG signal source in the region of interest (a region of the visual cortex in the pleasant image expectation state). FIG. 12C shows a result of a time-frequency analysis that was carried out on a signal of the EEG signal source in the region of interest (a region of the visual cortex in the unpleasant image expectation state). FIG. 12D shows the difference between the signals obtained in the pleasant image expectation state and the unpleasant image expectation state. In FIG. 12D, the region with a significant difference between them is encircled. Other regions had no difference. These EEG measurement results reveal that reactions in the α bands of the visual cortex were involved in the pleasant image expectation.

3. Configuration of Multi-Axis Sensibility Model by Human Type

The present inventors have found it is necessary to obtain actual measurements of the axes of the sensibility and specify the relationship among the axes contributing to the sensibility. Based on these findings, the present inventors have integrated a subjective psychological axial model of the sensibility and a neurophysiological index in the following manner for quantification of the sensibility.

Sensibility=[Subjective Psychological Axial Model]×[Neurophysiological Index]=a×EEGₚₗₑₐₛᵤᵣₑ+b×EEG_{activation}+c×EEG_{anticipation} (Formula 1)

where the subjective psychological axial model is composed of multiple axes each of which has own weighting coefficients (a, b, c), and the neurophysiological index represents the values (EEGₚₗₑₐₛᵤᵣₑ, EEG_{activation}, EEG_{anticipation}) of the axes based on the results of EEG measurement.

When Formula 1 is generalized, the degree of the sensibility is expressed by the following formula.

Sensibility=Σp×(Σq×x) (Formula 2)

where x is at least one feature value (e.g., time-frequency spectrum) extracted from neurophysiological data (e.g., a neural nerve activities grasped from the EEG) measured with a neural activity measuring apparatus (e.g., electroencephalograph), q is a weighting coefficient of the at least one feature value, (Σq×x) is at least one neurophysiological index (e.g., each of the pleasant/unpleasant axis, the activation/deactivation axis, and the anticipation axis in the multi-axis sensibility model) relating to the sensibility, and p is a weighting coefficient of the at least one neurophysiological index.

Moreover, the present inventors found, from experiments hitherto conducted, that a more accurate result of sensibility evaluation is obtained by classifying people into several types (human types) based on traits such as gender, age, and character, and applying a multi-axis sensibility model optimized for the human type of each person than by applying an average single multi-axis sensibility model to everyone. Thus, a configuration procedure of multi-axis sensibility models by human types according to one embodiment of the present disclosure will be described below.

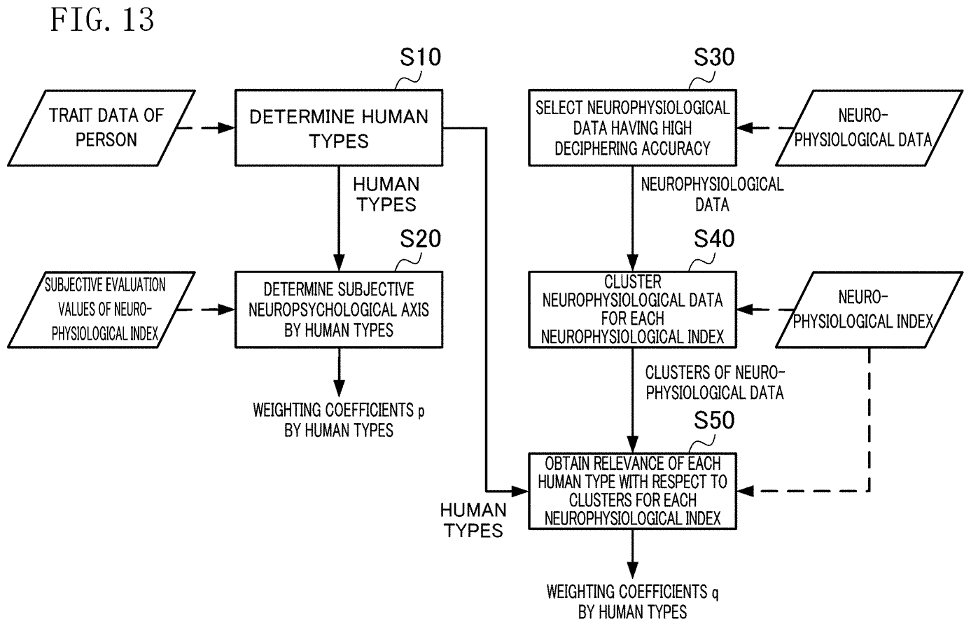

FIG. 13 shows a block flow diagram illustrating the configuration procedure of the multi-axis sensibility models by the human types. Schematically, trait information items (such as information of gender, age, and character) representing various traits of people are first clustered to determine human types for classification of the traits of the people (S10). Then, a subjective evaluation experiment relating to sensibility evaluation is performed on participants to obtain an experiment result, and a subjective evaluation value of at least one neurophysiological index obtained from the experiment result is subjected to a regression analysis (e.g., linear regression analysis) by the human types determined in step S10 to calculate weighting coefficients p (see Formula 2) by the human types (S20). For each of the at least one neurophysiological index, among neurophysiological data of the participants measured in the subjective evaluation experiment, selected are neurophysiological data having statistical significance to the each of the at least one neurophysiological index (S30). Moreover, for each of the at least one neurophysiological index, the neurophysiological data selected are clustered to determine clusters of the neurophysiological data (S40). Furthermore, for each of the at least one neurophysiological index, relevance of each of the human types with respect to the clusters is obtained to convert the relevance into the weighting coefficients q (see Formula 2) by the human types (S50). Determination of human types (S10), determination of a subjective psychological axial model (S20), selection of neurophysiological data (S30), statistical process of the neurophysiological data selected (S40), and determination of the at least one neurophysiological index for each human type (S50) will be sequentially described in detail below.

A. Determination of Human Type

People are classifiable into several groups, that is, human types in accordance with individual traits of the person. Examples of the trait information items for human type classification include objective trait information items such as gender, age or age group, residence, and nationality of a person and subjective trait information items such as thought, preference, sense of values, world view, and cognitive tendency of the person. For the human type classification, any one of the trait information items may be used, or a combination of the trait information items may be used. In the following description, an example will be described in which the human type classification is performed based on the character, which is one of the subjective trait information items.

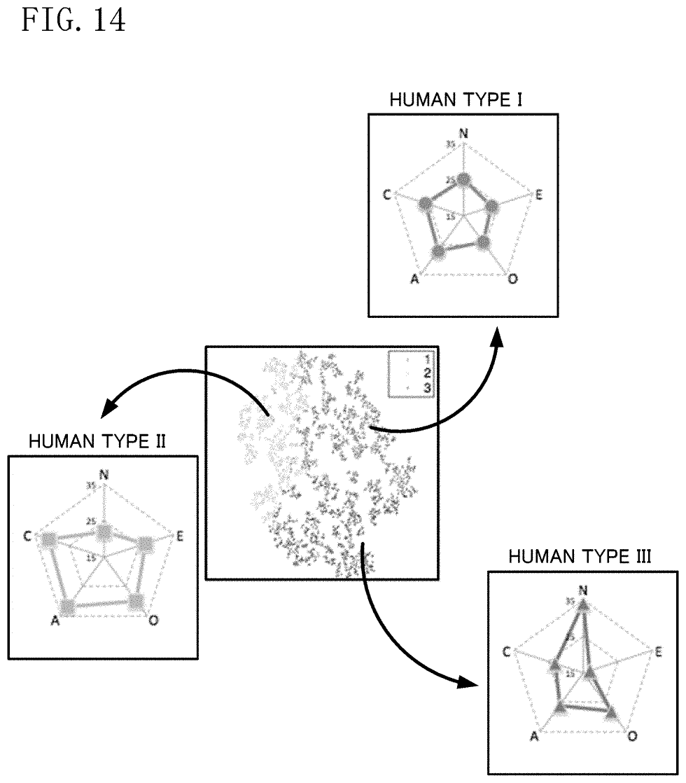

A Big Five personality assessment test for assessment of the character of a person based on a combination of five factors, namely, neuroticism, extraversion, openness, agreeableness, and conscientiousness was conducted to two groups of participants (3046 participants and 3140 participants) in an age range from 18 to 79. Then, results of the Big Five personality assessment test conducted to these two groups were clustered by k-means or the like. To determine the number of clusters to be extracted, one of statistical standard techniques such as Gap statistics was further applied. As a result, participants in each group were classifiable into three human types each of which are common between the two groups. FIG. 14 is a view schematically illustrating examples of three human types determined by the clustering of the five factors of character traits.

Note that the above-described determination of the human types is a mere example, and the number of participant groups and the number of participants in each patient group are not limited to the above-described numbers.

B. Determination of Subjective Psychological Axial Model

A contribution ratio, i.e., weighting, of each axis using the subjective psychological axial model of the sensibility can be determined in the following manner.

(1) The experiment of presenting the participants (28 male and female students) with the pleasant/unpleasant stimulus images is carried out as described above. Each participant is urged to make a self-evaluation of the sensibility state of the brain during a 4-second interval (expectation state) between a point in time when the tone is emitted and a point in time when the image is presented. Note that a simple Big Five personality assessment test is conducted to the participants in advance to specify the human type of each of the participants. The human types of the participants include a mix of the three human types described above.

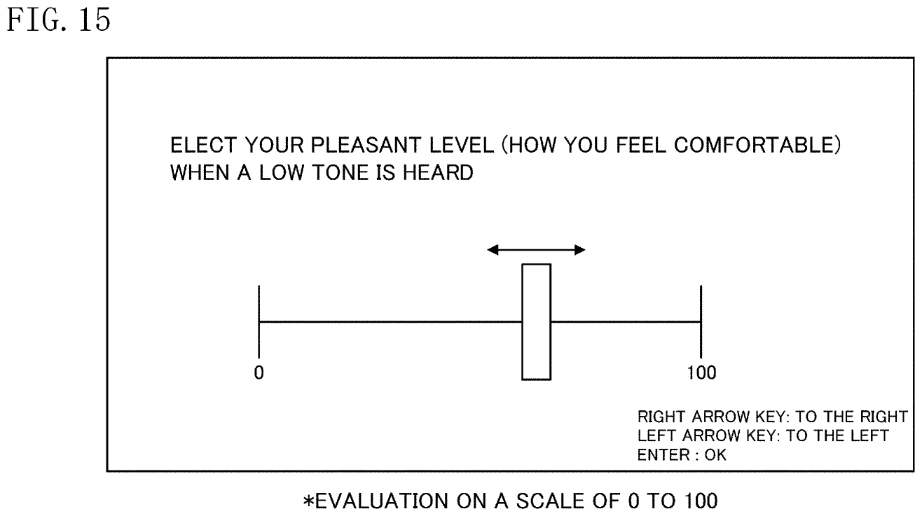

(2) The participants are urged to make an evaluation of an exhilaration (sensibility) level, a pleasure level (pleasure axis), an activity level (activation axis), and an anticipation level (anticipation axis) on a scale of 101 from 0 to 100 using Visual Analog Scale (VAS) under three different conditions (in the pleasant image expectation state, the unpleasant image expectation state, and the pleasant/unpleasant unexpectable state). FIG. 15 shows an example of the self-evaluation for the determination of the subjective psychological axial model, illustrating how the level of pleasure is evaluated when a low tone is emitted (in which a condition pleasant image was expected). Each participant moves the cursor between 0 and 100 for the evaluation. As a result of the evaluation, for example, subjective evaluation values of: exhilaration=73; pleasure=68; activation=45; and anticipation=78 are obtained from one of the participants in the pleasant image expectation state.

(3) Coefficients of the subjective psychological axial model are calculated through linear regression based on the subjective evaluation values obtained from all the participants belonging to respective human types under the three conditions. As a result, the following sensibility evaluation formulae based on the subjective psychological axial model are obtained by the human types.

Human type I: Sensibility=0.58×Subjectiveₚₗₑₐₛᵤᵣₑ+0.12×Subjective_{activation}+0.32×Subjective_{anticipation} (Formula 3)

Human type II: Sensibility=0.69×Subjectiveₚₗₑₐₛᵤᵣₑ+0.04×Subjective_{activation}+0.26×Subjective_{anticipation} (Formula 4)

Human type III: Sensibility=0.21×Subjectiveₚₗₑₐₛᵤᵣₑ+0.19×Subjective_{activation}+0.60×Subjective_{anticipation} (Formula 5)

where the subjectiveₚₗₑₐₛᵤᵣₑ, the subjective_{activation}, and the subjective_{anticipation} are values of the pleasure level, activation level, and anticipation level evaluated by the participants.

(4) The Subjectiveₚₗₑₐₛᵤᵣₑ, Subjective_{activation}, and Subjective_{anticipation} of the subjective psychological axial model respectively correspond to the EEGₚₗₑₐₛᵤᵣₑ, EEG_{activation}, and EEG_{anticipation} of the neurophysiological index. Thus, the weighting coefficients of the axes of the subjective psychological axial model calculated from the linear regression of the subjective evaluation values can be used as weighting coefficients (weighting coefficients p in Formula 2) of the EEGₚₗₑₐₛᵤᵣₑ, EEG_{activation}, and EEG_{anticipation} of the neurophysiological index. If the weighting coefficients of the axes obtained from Formulae 3 or 5 are applied to Formula 1, the sensibility can be represented by the following formulae using the EEGₚₗₑₐₛᵤᵣₑ, the EEG_{activation}, and the EEG_{anticipation} which are measured from moment to moment.

Human type I: Sensibility=0.58×EEGₚₗₑₐₛᵤᵣₑ+0.12×EEG_{activation}+0.32×EEG_{anticipation} (Formula 6)

Human type II: Sensibility=0.69×EEGₚₗₑₐₛᵤᵣₑ+0.04×EEG_{activation}+0.26×EEG_{anticipation} (Formula 7)

Human type III: Sensibility=0.21×EEGₚₗₑₐₛᵤᵣₑ+0.19×EEG_{activation}+0.60×EEG_{anticipation} (Formula 8)

Specifically, the sensibility of exhilaration can be quantified as a form of numerical value by one of Formulae 6 to 8 corresponding to one's human type determined by the above mentioned method in “A. Determination of Human Type.”

C. Selection of Neurophysiological Data

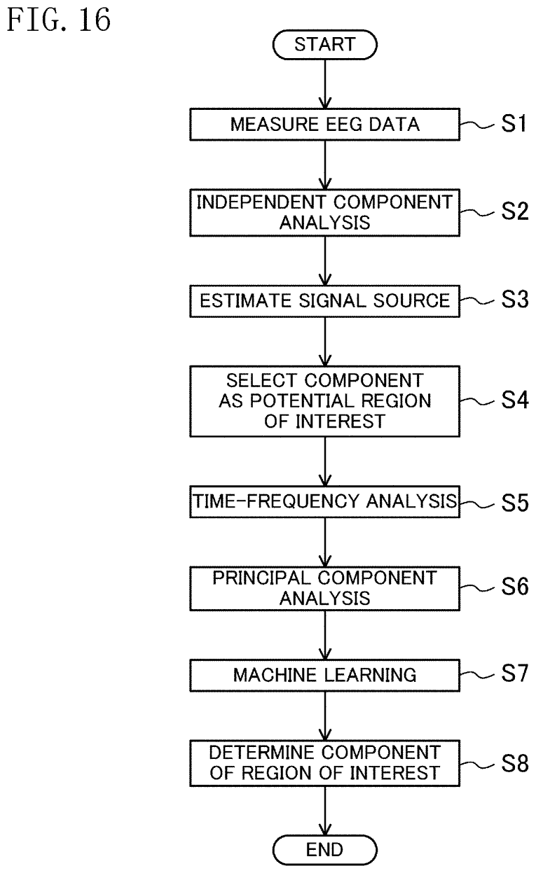

FIG. 16 shows a flow chart for selection of the independent components of the electroencephalogram in the region of interest and their frequency bands. For example, an image which evokes pleasant/unpleasant is presented as a visual stimulus to a subject, and electroencephalogram signals responding to the stimulus is measured (step S1). Noise derived from blink, movement of eyes, and myoelectric potential (artifact) is removed from the measured electroencephalogram signal.

An independent component analysis (ICA) is performed on the measured electroencephalogram signal to extract independent components (and signal sources of the components) (step S2). For example, when the electroencephalogram is measured with 32 channels, the corresponding number of independent components, i.e., 32 independent components, are extracted. As a result of the independent component analysis of the measured electroencephalogram, the positions of the signal sources are identified (step S3).

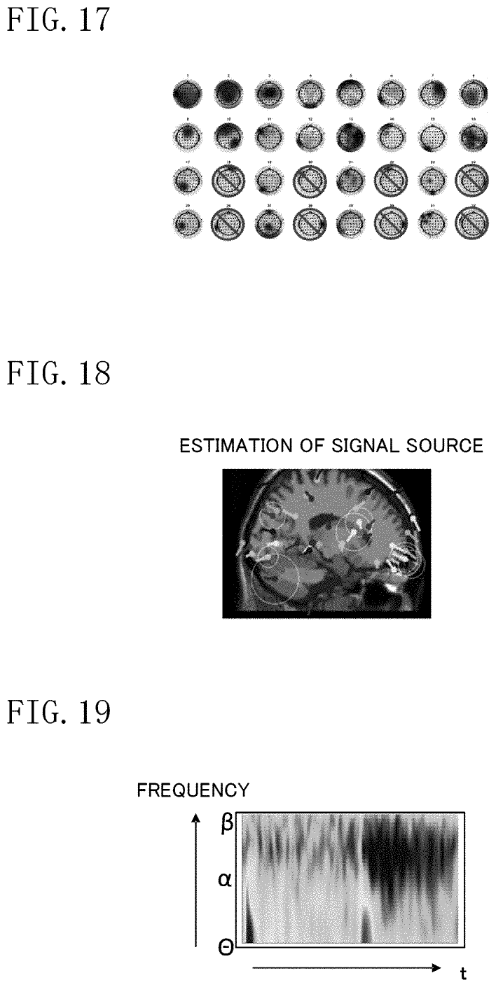

FIG. 17 shows components (electroencephalogram topographic images) representing signal intensity distributions of the independent components extracted through the independent component analysis carried out on the electroencephalogram signal in step S2. FIG. 18 shows a sagittal section of the brain on which estimated positions of the signal sources of the independent signal components are plotted.

For example, if the independent component related to the “pleasant” state is a target to be selected, the signal sources (independent components) present around the cingulate gyms can be selected as one of potential regions of interest (step S4). For example, 10 independent components are selected out of the 32 independent components for a person.

With respect to each of the signals (e.g., 10 independent components) from the signal sources as the potential regions of interest, a time-frequency analysis is performed to calculate a power value at each time point and each frequency point (step S5). For example, 20 frequency points are set at each of 40 time points to calculate the power value at 800 points in total.

FIG. 19 shows a result of a time-frequency analysis that was carried out on the signals of the EEG signal sources in step S5. In the graph of FIG. 19, a vertical axis represents the frequency, and a horizontal axis the time. The frequency 3 is the highest, a is the second highest, and θ is the lowest. The intensity of the gray in the graph corresponds to the signal intensity. The result of the time-frequency analysis is actually shown in color, but the graph of FIG. 19 is shown in gray scale for convenience.

Then, a principal component analysis (PCA) is performed on each of the independent components gained through time-frequency decomposition, thereby narrowing each independent component into a principal component based on time and frequency bands (step S6). In this way, narrowing into the number of significant features is performed. For example, the features at the 800 points are dimensionally reduced to 40 principal components.

Discrimination learning is carried out on the narrowed time-frequency principal components using sparse logistic regression (SLR) (step S7). Thus, the principal component (time frequency) which contributes to the discrimination of the axis (e.g., the pleasant/unpleasant axis) of the independent component (signal source) is detected. For example, in measuring the subject's “pleasure,” it is determined that the θ band of the signal source in the region of interest is relevant. Further, the discrimination accuracy in the frequency band of the independent component can be calculated, for example, the accuracy of the discrimination between two choices of pleasure and displeasure is 70%.

Based on the calculated discrimination accuracy, the independent component and its frequency band with a significant discrimination rate is identified (step S8). Thus, among the 10 independent components as the potential regions of interest, for example, one or more independent components and their frequency bands are selected.

The procedure for measuring the pleasant/unpleasant feeling has been described above. In the similar procedure, the independent components of the electroencephalogram in the region of interest and their frequency bands are identified for the measurement of the active/inactive feeling and the anticipation. The results of the measurements reveal that the β band of the region of interest is involved in the activation/deactivation, and the θ to α bands are involved in the anticipation.

D. Statistical Process of Selected Neurophysiological Data

Selected neurophysiological data of all the participants are collected and clustered by Gaussian mixture model (GMM). To determine the number of clusters, the Bayesian information criterion or the like may be adopted. When the selected neurophysiological data are independent components of the electroencephalogram, a spatial weight vector of each of the independent components (the weighting value of each channel) is to be clustered.

For example, from 28 participants retained components were a group of 118 independent components of the electroencephalogram which have statistical significance to the neurophysiological index “pleasant/unpleasant”, a group of 128 independent components of the electroencephalogram which have statistical significance to the neurophysiological index “active/inactive”, and a group of 148 independent components of the electroencephalogram which have statistical significance to the neurophysiological index “anticipation”, and clustering of each of the groups into 7-9 clusters was possible. FIG. 20 shows a view schematic example of scattered dots, each dot representing one of independent components observed from a person, illustrating clusters of neurophysiological data relating to pleasant/unpleasant and their electroencephalogram topographic images. A scatter plot in the figure shows 118 independent components. Note that each independent component is clustered within multi-dimensional data (32 dimensions because of 32 channels data was used in this case) but is, for the sake of convenience, expressed in two dimensions by using t-SNE (t-distributed Stochastic Neighbor Embedding). Nine electroencephalogram topographic images in the figure representatively show nine clusters.

E. Determination of Neurophysiological Index by Human Type

As shown in Formula 2, the neurophysiological index can be expressed by (Σq×x), and a value different for each human type is applied to the weighting coefficients q. For example, the neurophysiological index “EEGₚₗₑₐₛᵤᵣₑ” is calculated based on the following formulae by the human types.

Human type I: EEGₚₗₑₐₛᵤᵣₑ=q₁⁽¹⁾×x₁+q₂⁽¹⁾×x₂+ . . . +qₙ⁽¹⁾×xₙ (Formula 9)

Human type II: EEGₚₗₑₐₛᵤᵣₑ=q₁⁽²⁾×x₁+q₂⁽²⁾×x₂+ . . . +qₙ⁽²⁾×xₙ (Formula 10)

Human type III: EEGₚₗₑₐₛᵤᵣₑ=q₁⁽³⁾×x₁+q₂⁽³⁾×x₂+ . . . +qₙ⁽³⁾×xₙ (Formula 11)

In order to obtain the weighting coefficients q by the human types, relevance of each human type with respect to the clusters is obtained for each neurophysiological index. The relevance may be obtained by a statistical analysis method such as a corresponding analysis. Then, the obtained relevance is converted into the weighting coefficients q by the human types.

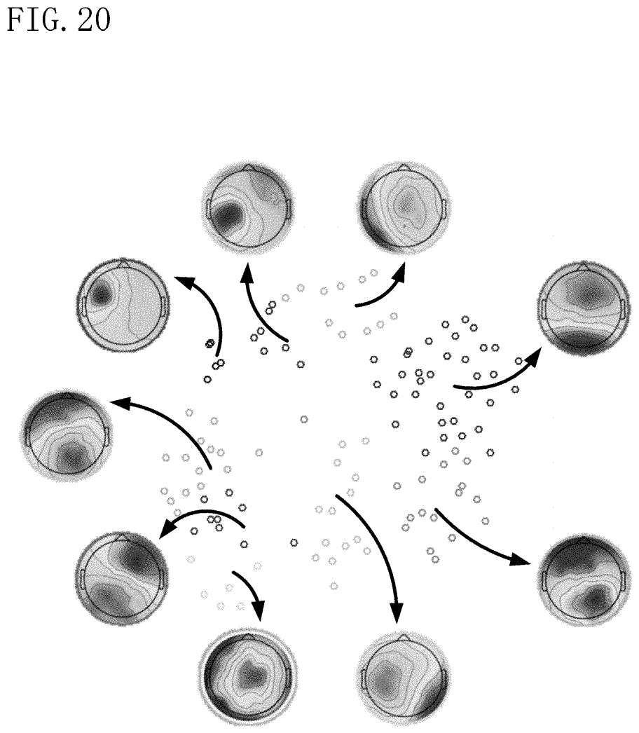

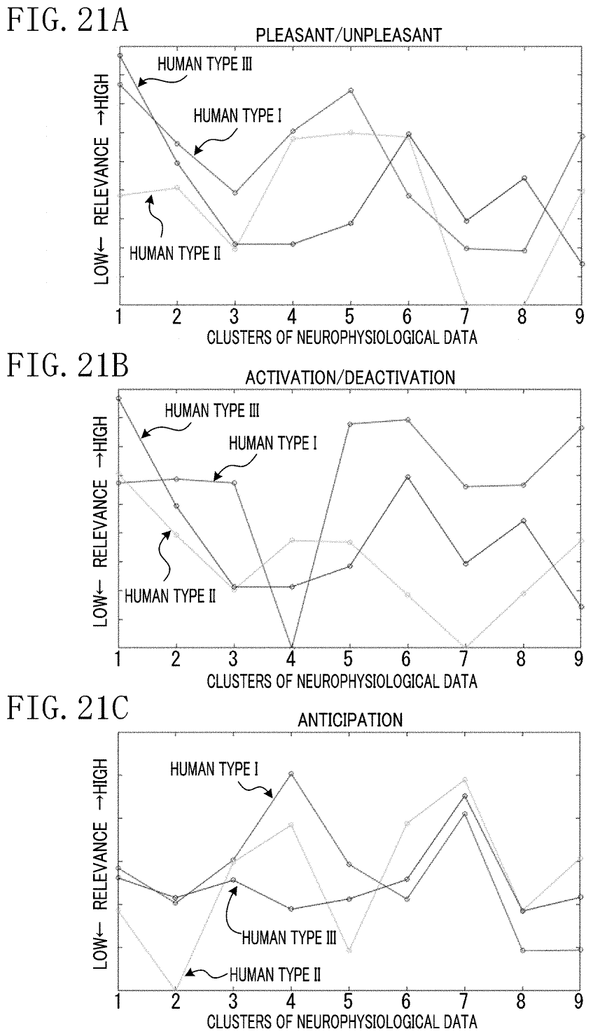

FIGS. 21A to 21C show graphs illustrating relevance of the human types with respect to the clusters for a corresponding one of neurophysiological indices of pleasant/unpleasant, activation/deactivation, and anticipation, respectively. For example, regarding the neurophysiological index “pleasant/unpleasant”, the contribution of a fifth cluster of the neurophysiological data is relatively high in people of human type I and is relatively low in people of human type III. Thus, the contribution to the neurophysiological index may differ depending on the human types even for the same cluster.

4. Real-Time Evaluation of Sensibility

Next, the sensibility evaluation apparatus will be described which is configured to evaluate sensibility of a user in real time based on the multi-axis sensibility models configured by the human types in accordance with the procedure described above.

(Embodiment of Sensibility Evaluation Apparatus)

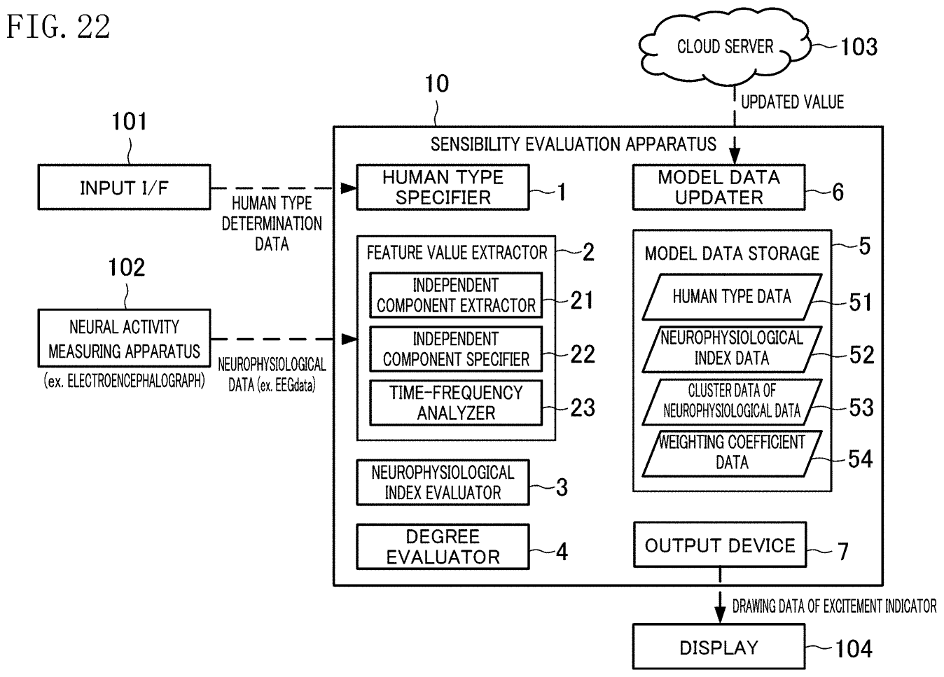

FIG. 22 is a block diagram illustrating a sensibility evaluation apparatus according to an embodiment of the present disclosure. A sensibility evaluation apparatus 10 includes a human type specifier 1, a feature value extractor 2, a neurophysiological index evaluator 3, a degree evaluator 4, model data storage 5, a model data updater 6, and an output device 7. Note that the sensibility evaluation apparatus 10 is configurable by installing a sensibility evaluation program on a personal computer or installing a sensibility evaluation application on a smartphone or a tablet terminal.

The human type specifier 1 specifies, among predetermined human types obtained by classifying traits of people, a human type to which a user subjected to sensibility evaluation belongs. Note that the human types are, for example, human type I to human type III described above, and the presence of the three human types is stored as human type data 51 in the model data storage 5. To specify the human type of a user, a Big Five personality assessment test may be conducted by using a paper medium or the like, and answers to the test may be input via an input interface 101 such as a keyboard, a mouse, or a touch panel to the sensibility evaluation apparatus 10, or a personality assessment application or the like may be executed in the sensibility evaluation apparatus 10 to perform a simple human type assessment. Note that the human type specifier 1 may store the specified human type associated with the user in memory (not shown). Thus, when the user logs in to the sensibility evaluation apparatus 10 the next and succeeding times, the human type specifier 1 can specify the human type of the user from login information without performing a personality assessment test.

The feature value extractor 2 receives the neurophysiological data of a user measured with a neural activity measuring apparatus 102 to specify, for each of the at least one neurophysiological index, among the neurophysiological data received, neurophysiological data which belong to predetermined clusters of the neurophysiological data received and which have statistical significance to the each of the at least one neurophysiological index, and extracts the at least one feature value from the neurophysiological data specified. For example, when the neural activity measuring apparatus 102 includes an electroencephalograph, and the neurophysiological data include electroencephalogram signals, the feature value extractor 2 includes an independent component extractor 21, an independent component specifier 22, and a time-frequency analyzer 23.

The independent component extractor 21 receives electroencephalogram signals (neurophysiological data) of a user measured by the electroencephalograph (neural activity measuring apparatus 102), and performs an independent component analysis on the electroencephalogram signals to extract independent components. Note that the electroencephalograph used may be a high-density electrode electroencephalograph including a large number of channels or may be a wearable electrode electroencephalograph including one or more channels. When the electroencephalograph is not compatible with artifact removal, the independent component extractor 21 performs a process of removing noise such as artifact on the electroencephalogram signals received from the electroencephalograph.

The independent component specifier 22 specifies, for each of the at least one neurophysiological index, among the independent components extracted, independent components belonging to the clusters. The presence of, for example, three neurophysiological indices (pleasant/unpleasant, active/inactive, and anticipation) is stored as neurophysiological index data 52 in the model data storage 5. Moreover, as described above, each neurophysiological index may include, for example, nine clusters (first to ninth clusters), which is stored as the cluster data 53 of the neurophysiological data in the model data storage 5. In this example, the independent component specifier 22 refers to the neurophysiological index data 52 and the cluster data 53 stored in the model data storage 5 so as to specify, for each of the three neurophysiological indices, among the independent components extracted, independent components belonging to the nine clusters.

The time-frequency analyzer 23 performs a time-frequency analysis on the independent components specified to calculate a time-frequency spectrum, and from the time-frequency spectrum calculated, the time-frequency analyzer 23 extracts a spectrum intensity in a frequency band of interest as a feature value. For example, since it is known that the frequency band of interest in the independent components according to the neurophysiological index “pleasant/unpleasant” is the θ band, the time-frequency analyzer 23 extracts, as the feature value, a spectrum intensity in the band from the independent components specified.

The neurophysiological index evaluator 3 selects, for each of the at least one neurophysiological index, a weighting coefficient q (see Formula 2) corresponding to the human type of the user from predetermined weighting coefficients q (see Formula 2) by the predetermined human types and applies the weighting coefficient q selected to the at least one feature value extracted to evaluate the each of the at least one neurophysiological index. The weighting coefficient q is stored as the weighting coefficient data 54 in the model data storage 5. For example, in the case of three neurophysiological indices (pleasant/unpleasant, active/inactive, and anticipation), three human types (Human type I to Human type III), and nine clusters (first to ninth clusters of the neurophysiological data) per neurophysiological index, a numerical value representing the total number of weighting coefficients q, namely, 3×3×9=81, is stored in the mode data storage 5. In this example, the neurophysiological index evaluator 3 reads, for each of the pleasant/unpleasant, activation/deactivation, and anticipation, nine weighting coefficients q corresponding to the human type of the user from the model data storage 5, and the neurophysiological index evaluator 3 applies the weighting coefficients q to respective nine feature values to evaluate the neurophysiological indices.

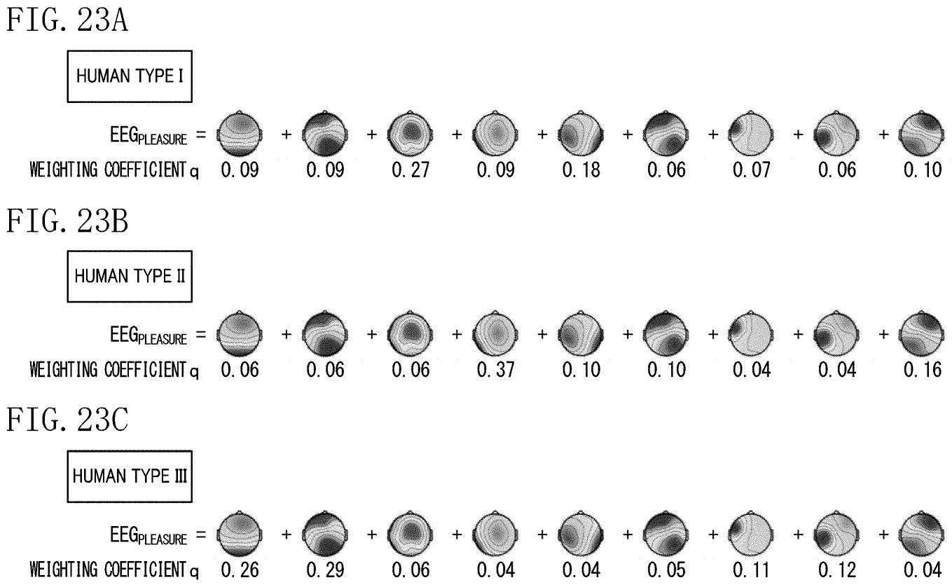

FIGS. 23A to 23C show topographical images of selected independent components of interest relevant to pleasant/unpleasant and their corresponding weighting coefficients and schematically illustrating Formula 9 to Formula 11, respectively. The electroencephalogram topographic images in the figures show the feature values (x in Formula 9 to Formula 11) extracted from the independent components belonging to the clusters. As shown in FIGS. 23A to 23C, different (or in some cases, the same) weighting coefficients q depending on the human types are applied to the feature values even when the feature values relate to the same cluster, and thereby the neurophysiological indices “EEGₚₗₑₐₛᵤᵣₑ” by the human types are calculated. Note that similarly to the case of the pleasant/unpleasant, for the neurophysiological indices “EEG_{activation}” and “EEG_{anticipation}”, different (or in some cases, the same) weighting coefficients q depending on the human types are applied to the feature values even when the feature values relate to the same cluster, and thereby the neurophysiological indices “EEG_{activation}” and “EEG_{anticipation}” by the human types are calculated.

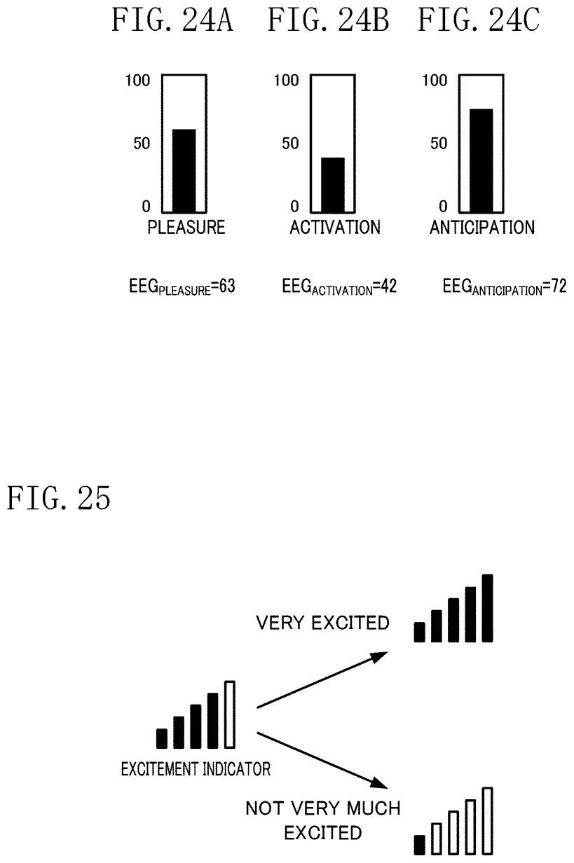

Each neurophysiological index is expressed by a numerical value, for example, from 0 to 100. FIGS. 24A to 24C are views schematically illustrating valued degrees of the pleasant/unpleasant, the activation/deactivation, and the anticipation, respectively. For example, as illustrated in FIGS. 24A to 24C, EEGₚₗₑₐₛᵤᵣₑ=63 is evaluated as the degree of the pleasant/unpleasant, EEG_{activation}=42 is evaluated as the degree of the activation/deactivation, and EEG_{anticipation}=72 is evaluated as the degree of the anticipation.

Referring back to FIG. 22, the degree evaluator 4 selects, from predetermined weighting coefficients p (see Formula 2) by the human types, a weighting coefficient p corresponding to the human type of a user and applies the weighting coefficient p selected to the neurophysiological index to evaluate the degree of the sensibility (see Formula 2). The weighting coefficient p is stored as the weighting coefficient data 54 in the model data storage 5. For example, in the case of three neurophysiological indices (pleasant/unpleasant, active/inactive, and anticipation), and three human types (Human type I to Human type III), a numerical value representing the total number of weighting coefficients p (nine weighting coefficients shown in Formula 6 to Formula 8), namely, 3×3=9, is stored in the model data storage 5. In this example, the neurophysiological index calculator 4 reads three weighting coefficients p corresponding to the human type of the user from the model data storage 5, and applies the weighting coefficients p to the respective three neurophysiological indices to evaluate the degree of the sensibility.

The model data storage 5 stores data of the multi-axis sensibility model such as the human type data 51, the neurophysiological index data 52, the cluster data 53, and the weighting coefficient data 54 described above. Note that the model data storage 5 desirably includes flush memory or the like in which data is rewritable. This is to be able to update model data each time the multi-axis sensibility model is improved.

The model data updater 6 receives, for example, from a cloud server 103, the latest model data of the multi-axis sensibility model (updated value of the data) to update the data stored in the model data storage 5. The multi-axis sensibility model by the human type is not fixed but is always updated while participants and sample data are accumulated. The cloud server 103 stores model data of such an updated multi-axis sensibility model and sends the update value of the data from the cloud server 103 to the sensibility evaluation apparatus 10 at an appropriate timing, which enables the sensibility evaluation apparatus 10 to perform sensibility evaluation based on the latest multi-axis sensibility model.

The output device 7 outputs the degree evaluated so that a person recognizes the degree evaluated. The output device 7 generates drawing data of an excitement indicator as an example of, for example, a BEI from the degree evaluated and displays the excitement indicator on a display 104. FIG. 25 illustrating a display example of an excitement indicator. For example, the excitement indicator represents the senses of excitement (the degree evaluated) of a user in a bar graph. Thus, visualizing the degree evaluated enables the variation of sensibility of a user to be intuitively grasped in real time.

As described above, the sensibility evaluation apparatus 10 according to the present embodiment requires no configuration of model optimized for each user from the beginning to perform sensibility evaluation and enables everyone to immediately perform the sensibility evaluation. Moreover, in the sensibility evaluation apparatus 10, an average simple standard model applicable to all people is not applied but the sensibility evaluation is performed based on a model according to the human type of the user. Therefore, it is possible to obtain a more accurate sensibility evaluation result.

Note that in the neurophysiological index evaluator 23, feature values of all clusters (in the above example, nine feature values) do not have to be taken into consideration, but only some of the feature values (for example, top three feature values) may be taken into consideration. In other words, weighting coefficients q corresponding to some of the clusters may be set to zero. Thus, in the time-frequency analyzer 23, ignorable feature values of the clusters no longer have to be extracted, and the amount of data to be subjected to an analysis process is thus reduced, which enables evaluation speed to be accelerated and power consumption to be reduced.

Moreover, in the above example, the electroencephalogram signals have been described as examples of the neurophysiological data, but data of fMRI and/or fNIRS other than the electroencephalogram signals may be used. Alternatively, physiological data such as heart rate, blood pressure, pulse (photoplethysmogram), and the like other than brain signals may be used.

### Other Embodiments

It has been described that the sensibility evaluation apparatus 10 is realizable by installing dedicated software on a personal computer, a smartphone, or the like. However, degree evaluation requires relatively complicated computation. Therefore, it is concerned that in a portable terminal such as a smartphone, a tablet terminal, or the like, computation capacity may be insufficient, or power consumption may impede its proper function. Thus, the sensibility evaluation apparatus 10 may be installed on the cloud server 103 having large computation capacity and may be realized as “Software as a Service” (SaaS).

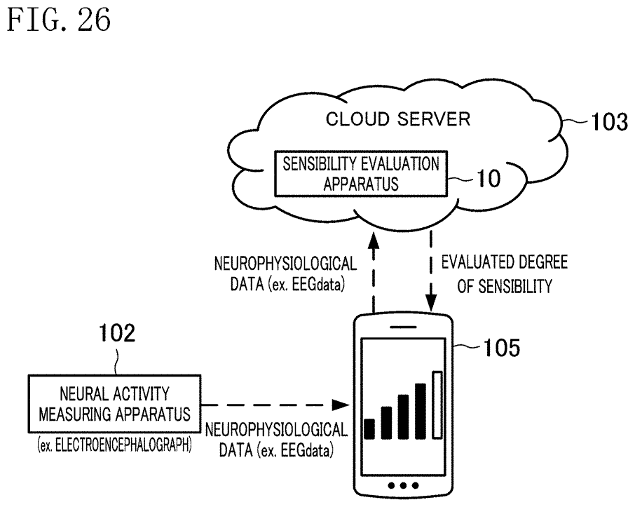

FIG. 26 is a view schematically illustrating an embodiment in which the sensibility evaluation apparatus is placed in a cloud environment. The sensibility evaluation apparatus 10 is arranged on the cloud server 103. A user can access the sensibility evaluation apparatus 10 on the cloud by using a portable terminal 105 such as a smartphone or a tablet terminal, or the like that has a capacity to access to the cloud. Specifically, the portable terminal 105 receives neurophysiological data of a user measured with the neural activity measuring apparatus 102 and transfers the neurophysiological data to the sensibility evaluation apparatus 10 on the cloud. Moreover, when data required to specify the human type of the user is input to the portable terminal 105, the portable terminal 105 transfers, to the sensibility evaluation apparatus 10, the data input. The sensibility evaluation apparatus 10 processes the neurophysiological data of the user transferred from the portable terminal 105 to evaluate the degree of the sensibility and transmits the degree to the portable terminal 105. The portable terminal 105 accordingly processes the degree transmitted from the sensibility evaluation apparatus 10 into an image and displays the image on a display of the portable terminal 105.

As described above, installing the sensibility evaluation apparatus 10 on the cloud server 103 enables the portable terminal 105 having a relatively small computation capacity to display a highly accurate sensibility evaluation result in real time without applying a processing load to the portable terminal 105.

Note that when the sensibility evaluation apparatus 10 is arranged in the cloud environment, components included in the sensibility evaluation apparatus 10 do not have to be collectedly arranged on one server but may be distributed on servers.

As can be seen in the foregoing, embodiments have just been described as examples of the technique disclosed in the present disclosure. For this purpose, accompanying drawings and detailed description have been provided.

The components illustrated on the accompanying drawings and described in the detailed description include not only essential components that need to be used to overcome the problem, but also other unessential components that do not have to be used to overcome the problem. Therefore, such unessential components should not be taken for essential ones, simply because such unessential components are illustrated in the drawings or mentioned in the detailed description.

The above embodiments, which have been described as examples of the technique of the present disclosure, may be altered or substituted, to which other features may be added, or from which some features may be omitted, within the range of claims or equivalents to the claims.

What is claimed is:

1. A sensibility evaluation apparatus for evaluating a degree of sensibility of a person, the degree being represented by Σp×(Σq×x), where x is at least one feature value extracted from neurophysiological data measured with a neural activity measuring apparatus, q is a weighting coefficient of the at least one feature value, (Σq×x) is at least one neurophysiological index relating to the sensibility of the person, and p is a weighting coefficient of the at least one neurophysiological index, the sensibility evaluation apparatus comprising:
a specifier configured to specify, among predetermined human types which are obtained by classifying traits of people, a human type of a user subjected to evaluation of sensibility;
an extractor configured to receive the neurophysiological data of the user measured with the neural activity measuring apparatus to specify, for each of the at least one neurophysiological index, among the neurophysiological data received, neurophysiological data which belong to predetermined clusters of the neurophysiological data received and which have statistical significance to the each of the at least one neurophysiological index, and extract the at least one feature value from the neurophysiological data specified;
a first evaluator configured to select, for each of the at least one neurophysiological index, a weighting coefficient q corresponding to the human type of the user from predetermined weighting coefficients q by the predetermined human types and apply the weighting coefficient q selected to the at least one feature value extracted to evaluate the each of the at least one neurophysiological index; and
a second evaluator configured to select a weighting coefficient p corresponding to the human type of the user from predetermined weighting coefficients p by the predetermined human types and apply the weighting coefficient p selected to the each of the at least one neurophysiological index evaluated to evaluate the degree.

2. The sensibility evaluation apparatus of claim 1, further comprising:
data storage configured to store data of the predetermined human types, the at least one neurophysiological index, the predetermined clusters, and the predetermined weighting coefficients p and q; and
a data updater configured to receive updated values for the data to update the data.

3. The sensibility evaluation apparatus of claim 1, further comprising
an output device configured to output the degree evaluated so that a person recognizes the degree evaluated.

4. The sensibility evaluation apparatus of claim 1, wherein
the at least one neurophysiological index includes three neurophysiological indices representing pleasant/unpleasant, activation/deactivation, and anticipation.

5. The sensibility evaluation apparatus of claim 1, wherein
the neural activity measuring apparatus includes an electroencephalograph,
the neurophysiological data include electroencephalogram signals, and
the extractor includes
an independent component extractor configured to receive the neurophysiological data of the user measured with the neural activity measuring apparatus and perform an independent component analysis on the neurophysiological data to extract independent components,
an independent component specifier configured to specify, for each of the at least one neurophysiological index, among the independent components extracted, independent components belonging to each of the predetermined clusters, and
an analyzer configured to perform a time-frequency analysis on the independent components specified to calculate a time-frequency spectrum and extract, from the time-frequency spectrum calculated, a spectrum intensity in a frequency band of interest as the at least one feature value.

6. A sensibility evaluation method for evaluating a degree of sensibility of a person, the degree being represented by Σp×(Σq×x), where x is at least one feature value extracted from neurophysiological data measured with a neural activity measuring apparatus, q is a weighting coefficient of the at least one feature value, (Σq×x) is at least one neurophysiological index relating to the sensibility of the person, and p is a weighting coefficient of the at least one neurophysiological index, the sensibility evaluation method comprising:
specifying, among predetermined human types which are obtained by classifying traits of people, a human type of a user subjected to evaluation of sensibility;
receiving the neurophysiological data of the user measured with the neural activity measuring apparatus to specify, for each of the at least one neurophysiological index, among the neurophysiological data received, neurophysiological data which belong to predetermined clusters of the neurophysiological data received and which have statistical significance to the each of the at least one neurophysiological index, and extracting the at least one feature value from the neurophysiological data specified;
selecting, for each of the at least one neurophysiological index, a weighting coefficient q corresponding to the human type of the user from predetermined weighting coefficients q by the predetermined human types, and applying the weighting coefficient q selected to the at least one feature value extracted to evaluate the each of the at least one neurophysiological index; and
selecting a weighting coefficient p corresponding to the human type of the user from predetermined weighting coefficients p by the predetermined human types, and applying the weighting coefficient p selected to the each of the at least one neurophysiological index evaluated to evaluate the degree.

7. The method of claim 6, further comprising:
receiving updated values of data of the predetermined human types, the at least one neurophysiological index, the predetermined clusters, and the predetermined weighting coefficients p and q to update the data.

8. The method of claim 6, further comprising:
outputting the degree evaluated so that a person recognizes the degree evaluated.

9. The method of claim 6, wherein
the at least one neurophysiological index includes three neurophysiological indices representing pleasant/unpleasant, activation/deactivation, and anticipation.

10. The method of claim 6, wherein
the neural activity measuring apparatus includes an electroencephalograph,
the neurophysiological data include electroencephalogram signals, and
the extracting of the at least one feature value includes
receiving the neurophysiological data of the user measured with the neural activity measuring apparatus and performing an independent component analysis on the neurophysiological data to extract independent components,
specifying, for each of the at least one neurophysiological index, among the independent components extracted, independent components belonging to each of the predetermined clusters, and
performing a time-frequency analysis on the independent components specified to calculate a time-frequency spectrum and extract, from the time-frequency spectrum calculated, a spectrum intensity in a frequency band of interest as the at least one feature value.

11. A method for configuring a multi-axis sensibility model representing a degree of sensibility of a person by Σp×(Σq×x), where x is at least one feature value extracted from neurophysiological data measured with a neural activity measuring apparatus, q is a weighting coefficient of the at least one feature value, (Σq×x) is at least one neurophysiological index relating to the sensibility of the person, and p is a weighting coefficient of the at least one neurophysiological index, the method comprising:
clustering qualitative data representing traits of the person to determine human types for classification of the traits of the person;
performing a regression analysis by the human types on subjective evaluation values of the at least one neurophysiological index obtained by performing a subjective evaluation experiment on participants to calculate weighting coefficients p by the human types;
selecting, for each of the at least one neurophysiological index, among neurophysiological data of the participants measured in the subjective evaluation experiment, neurophysiological data having statistical significance to the each of the at least one neurophysiological index;
clustering, for each of the at least one neurophysiological index, the neurophysiological data selected to determine clusters of the neurophysiological data; and
obtaining, for each of the at least one neurophysiological index, relevance of each of the human types with respect to the clusters to convert the relevance into the weighting coefficients q by the human types.

12. The method of claim 11, wherein
the at least one neurophysiological index includes three neurophysiological indices representing pleasant/unpleasant, activation/deactivation, and anticipation.

13. The method of claim 11, wherein
the neurophysiological data include electroencephalogram signals, and
the selecting of the neurophysiological data includes
performing an independent component analysis on neurophysiological data of the participants measured in the subjective evaluation experiment to extract independent components, and
selecting, for each of the at least one neurophysiological index, an independent component having statistical significance to the neurophysiological index from the independent components extracted.
